(19) United States
(12) Patent Application Publication
(10) Pub. No.: US 2009/0297459 A1
(43) Pub. Date: Dec. 3, 2009

(54) TRANSPARENT SILICA GEL/PRECIPITATED SILICA COMPOSITE MATERIALS FOR DENTIFRICES

(76) Inventors: Duen-Wu Hua, Edgewood, MD (US); Patrick Donald McGill, Darlington, MD (US); William C. Fultz, Rising Sun, MD (US)

Correspondence Address: J M HUBER CORPORATION, 1000 Parkwood Circle, Ste. 1000, PATENT DEPARTMENT, Atlanta, GA, 30339, US

(21) Appl. No.: 12/468,910

(22) Filed: May 20, 2009

Related U.S. Application Data
(60) Provisional application No. 61/058,409, filed on Jun. 3, 2008.

(51) Int. Cl. A61K 8/25; A61Q 11/00

(52) U.S. Cl. 424/49

(57) ABSTRACT

A gel/precipitate silica composite for use in a dentifrice composition has a maximum light transmission of at least 25% within a refractive index range of from about 1.432 to about 1.455; a relative flavor availability as compared to silica sand of at least 50%; a CTAB of less than about 40; and, when incorporated into a dentifrice composition in an amount of 20% by weight, said dentifrice has a RDA (Relative Dentin Abrasion) value of at most 130; a PCR (Pellicle Cleaning Ratio):RDA ratio of from 0.7 to 1.3; and a haze value after 24 hours of less than about 50%.

### CORRELATED APPLICATIONS

The present application claims the benefit of priority of U.S. Provisional Patent Application No. 61/058,409, filed Jun. 3, 2008, entitled Silica Materials for Dentrifices”, the disclosure of which is herein incorporated by reference in its entirety.

### TECHNICAL FIELD

This invention relates to silica gel and precipitated silica composite materials, and more particularly, to such composite materials having properties suitable for dentifrice applications.

### BACKGROUND

An abrasive substance has been included in conventional dentifrice compositions in order to remove various deposits, including pellicle film, from the surface of teeth. Pellicle film is tightly adherent and often contains brown or yellow pigments which impart an unsightly appearance to the teeth. While cleaning is important, the abrasive should not be so aggressive so as to damage the teeth. Ideally, an effective dentifrice abrasive material maximizes pellicle film removal while causing minimal abrasion and damage to the hard tooth tissues. Consequently, among other things, the performance of the dentifrice is highly sensitive to the extent of abrasion caused by the abrasive ingredient.

Synthetic low-structure silicas have been utilized for such a purpose due to the effectiveness such materials provide as abrasives, as well as low toxicity characteristics and compatibility with other dentifrice components, such as sodium fluoride, as one example. When preparing synthetic silicas, the objective is to obtain silicas which provide maximal cleaning with minimal impact to the hard tooth surfaces. Dental researchers are continually concerned with identifying abrasive materials that meet such objectives.

Synthetic high-structure silicas have been utilized as thickening agents for dentifrices and other like paste materials in order to supplement and modify the rheological properties for improved control, such as viscosity build, stand up, brush sag, and the like. For toothpaste formulations, for example, there is a need to provide a stable paste that can meet a number of consumer requirements, including, and without limitation, the ability to be transferred out of a container (such as a tube) via pressure (i.e., squeezing of the tube) as a dimensionally stable paste and to return to its previous state upon removal of such pressure, the ability to be transferred in such a manner to a brush head easily and without flow out of the tube during and after such transference, the propensity to remain dimensionally stable on the brush prior to use and when applied to target teeth prior to brushing, and proper mouth feel based on consumer preferences.

Generally, dentifrices comprise a majority of a humectant (such as sorbitol, glycerin, polyethylene glycol, and the like) in order to permit proper contact with target dental subjects, an abrasive (such as precipitated silica) for proper cleaning and abrading of the pellicle film of the subject teeth, water, and other active components (such as fluoride-based compounds for anticaries benefits). The ability to impart proper rheological benefits to such a dentifrice is accorded through the proper selection and utilization of thickening agents (such as hydrated silicas, hydrocolloids, gums, and the like) to form a proper network of support to properly contain such important humectant, abrasive, and anticaries ingredients.

A number of water-insoluble, abrasive polishing agents have been used or described for dentifrice compositions. These abrasive polishing agents include natural and synthetic abrasive particulate materials. The generally known synthetic abrasive polishing agents include amorphous precipitated silicas and silica gels and precipitated calcium carbonate (PCC). Other abrasive polishing agents for dentifrices have included chalk, magnesium carbonate, dicalcium phosphate and its dihydrate forms, calcium pyrophosphate, zirconium silicate, potassium metaphosphate, magnesium orthophosphate, tricalcium phosphate, perlite, and the like.

Synthetically produced precipitated low-structure silicas, in particular, have been used as abrasive components in dentifrice formulations due to their cleaning ability, relative safeness, and compatibility with typical dentifrice ingredients, such as humectants, thickening agents, flavoring agents, anticaries agents, and so forth. As known, synthetic precipitated silicas generally are produced by the destabilization and precipitation of amorphous silica from soluble alkaline silicate by the addition of a mineral acid and/or acid gases under conditions in which primary particles initially formed tend to associate with each other to form a plurality of aggregates (i.e., discrete clusters of primary particles), but without coalescence into a three-dimensional gel structure. The resulting precipitate is separated from the aqueous fraction of the reaction mixture by filtering, washing, and drying procedures, and then the dried product is mechanically comminuted in order to provide a suitable particle size and size distribution. The silica drying procedures are conventionally accomplished using spray drying with a nozzle (e.g., tower or fountain), or wheel, flash drying, oven/fluid bed drying, and the like.

As it is, such conventional abrasive materials suffer to a certain extent from limitations associated with maximizing cleaning and minimizing dentin abrasion. The ability to optimize such characteristics in the past has been limited generally to controlling the structures of the individual components utilized for such purposes. Examples of modifications in precipitated silica structures for such dentifrice purposes are described within such publications as U.S. Pat. Nos. 3,967,563, 3,988,162, 4,420,312, and 4,122,161 to Wason, U.S. Pat. Nos. 4,992,251 and 5,035,879 to Aldcroft et al., U.S. Pat. No. 5,098,695 to Newton et al., and U.S. Pat. Nos. 5,891,421 and 5,419,888 to McGill et al. Modifications in silica gels have also been described within such publications as U.S. Pat. Nos. 5,647,903 to McGill et al., U.S. Pat. No. 4,303,641, to DeWolf, II et al., U.S. Pat. No. 4,153,680, to Seybert, and U.S. Pat. No. 3,538,230, to Pader et al.

Many of the aforementioned problems have been addressed by prior art references such as U.S. Pat. No. 7,267,814 (McGill et al.), U.S. Pat. No. 7,306,788 (McGill et al.), the disclosures of which are herein incorporated by reference in their entirety. These patents disclose unique gel/precipitated silica combinations that were prepare by in situ reaction and production techniques. The gel/precipitated silica composite (combination) produced according to these patents results in a safer abrasive that exhibits a significantly higher Pellicle Cleaning Ratio (further defined herein and referenced as “PCR”) level versus Relative Dentin Abrasion (further defined herein and referenced as “RDA”) level than has previously been provided within the dental silica industry.

Furthermore, the in situ process disclosed in these patents obviates the requirement to produce the gel materials and precipitate materials separately and then meter them out for proper target levels, which adds costs and process steps to the manufacturing procedure.

While patents such as U.S. Pat. No. 7,267,814 and U.S. Pat. No. 7,306,788 document a substantial accomplishment in obtaining high-cleaning, low abrasive silica, they do not address all of the dentifrice relevant functional characteristics of silica. In particular, these patents do not address the necessary optical properties to make the gel/precipitated silica combination useful for inclusion in transparent dentifrices. This is particularly important because transparent toothpaste products have become increasingly popular in recent years because of their greater appeal to some consumers and because they allow manufacturers to impart increased distinctiveness to their product.

However, preparing silica suitable for inclusion in high-water transparent toothpastes presents another challenge; it is necessary that the silica's refractive index closely matches the refractive index of the toothpaste matrix. Water generally has a far lower refractive index than silica and humectants, such as glycerin and sorbitol. Thus, as the toothpaste formulator increases the amount of water in the toothpaste (in order to reduce the concentration of the humectants and hence the formulation cost), it is necessary to provide a silica with a lower refractive index in order for the refractive index of the silica to match the refractive index of the high-water toothpaste formulation. This need for silica with a low refractive index may be met by use of low-structure silica. However, low-structure silica may complicate the production of transparent toothpaste because low-structure silica is more likely to have a low degree of light transmittance. When low-structure silica is incorporated into toothpaste, the toothpaste tends to have reduced transparency caused by the low degree of light transmittance of the low-structure silica.

Another important characteristic of silica for dental applications is its flavor compatibility. Flavor is a particularly important characteristic of a dentifrice and is very important to dentifrice manufacturers in order to impart positive impressions in the minds of consumers and distinguish their product from competitors. Accordingly, it is important that silica materials not interfere with the characteristics of a flavor nor absorb the flavor so as to diminish its potency.

Accordingly, there is a need in the art for a silica that has a functional performance profile that includes good cleaning, low abrasivity, improved flavor compatibility, and a relatively high degree of transmittance, even at an index of refraction that is sufficiently low so that the silica can be included in a transparent toothpaste composition having a relatively high concentration of water. It is to the provisions of such that the present invention is primarily directed.

### BRIEF SUMMARY OF THE INVENTION

The present invention relates to a gel/precipitate silica composite, wherein the composite exhibits a maximum light transmission of at least 25%, preferably at least 40%, within a refractive index range of from about 1.432 to about 1.455; a relative flavor availability as compared to silica sand of at least 50%; a CTAB of less than about 40; and, when incorporated into a dentifrice composition in an amount of 20% by weight, the dentifrice has a Relative Dentin Abrasion (RDA) value of at most 130, preferably of at most 120; a Pellicle Cleaning Ratio:Relative Dentin Abrasion (PCR:RDA) ratio of from 0.7 to 1.3; and a haze value after 24 hours of less than about 50%.

The present invention further relates to a dentifrice comprising the gel/precipitate composite (combination).

The present invention also relates to a method of producing a gel/precipitate silica composite, said method comprising the sequential steps of (a) admixing an electrolyte, an alkali silicate. and an acidulating agent to form a silica gel in a reaction medium; and, without first washing, modifying, or purifying said silica gel, and (b) subsequently introducing to said reaction medium comprising said silica gel of step (a) a sufficient amount of an alkali silicate and an acidulating agent to form a precipitated silica, thereby producing a gel/precipitate silica composite.

### DETAILED DESCRIPTION OF THE INVENTION

All parts, percentages and ratios used herein are expressed by weight unless otherwise specified. All documents cited herein are incorporated by reference.

It has now been found that modifications in the processes for producing in situ gel/precipitate silica composites can result in the production of gel/precipitate silica composites for use in dentifrice compositions that have a number of important functional characteristics including improved clarity, optical performance and flavor compatibility. In one embodiment these improved functional characteristics can be controlled by the use of an electrolyte and shearing forces, amongst other processing parameters. The terminology “in situ” is used herein to mean that in the process the precipitate formation stage follows the gel formation stage in the same reactor without modification in any way of the first produced silica gel. In other word, the first produced silica gel is not washed, purified, cleaned, etc. prior to commencement of the precipitate formation stage.

As disclosed in U.S. Pat. No. 7,306,788 and further provided for in the present invention, the specific in situ formed gel/precipitate silica composites exhibit very high levels of pellicle film cleaning properties with a significantly lower dentin abrasion for better dental protection. As was determined in U.S. Pat. No. 7,270,803 to McGill et al., an improved process for making such gel/precipitated silica composites incorporates a high shear treatment step after the initial gel production stage has been accomplished and during the precipitate formation stage resulting in gel/precipitated silica composites having improved abrasive properties and brightness characteristics. What has been discovered by the present invention to further improve upon the gel/precipitate silica composites is the importance of adding an electrolyte, such as sodium sulfate, to the reaction medium (silicate solution or water) during formation of the silica gel and, optionally, during formation of the precipitate. As a result, the material of the present invention offers not only the improved functional performance seen in previous prior art references (improved cleaning without a concomitant increase in dentin or enamel abrasion), but also improved flavor compatibility (reflected in the flavor characteristics and performance documented below) and a relatively high degree of transmittance, even at an index of refraction that is sufficiently low so that the silica can be included in a transparent toothpaste composition having a relatively high concentration of water.

This invention encompasses a method for producing in situ silica gels and precipitated silicas composites, which can be summarized by the following sequence of steps: a) admixing a sufficient amount of an electrolyte, an alkali silicate and an acidulating agent together to form a silica gel in a reaction medium; and b) subsequent to silica gel formation, optionally under high shear conditions, introducing to said reaction medium of step “a” a sufficient amount of an alkali silicate and an acidulating agent to form a precipitated silica, thereby producing a gel/precipitate silica composite.

An essential element of the present invention is that an electrolyte is introduced in step (a). Optionally, additional electrolyte may be introduced in step (b). The electrolyte that must be utilized in this inventive process may be any typical type of salt compound that dissociates easily in an aqueous environment. The alkali metal salts and alkaline earth metal salts are potentially preferred in this respect. More particularly, such compounds may be sodium salts, calcium salts, magnesium salts, potassium salts, and the like. Still more particularly, such compounds may be sodium sulfate, sodium chloride, calcium chloride, and the like. Most preferred is sodium sulfate, to be introduced either in powder form within the reaction or dissolved within the acid component prior to reaction with the silicate.

Encompassed as well within this invention is the product of such a process wherein the silica gel amount present therein is from 5 to 60% by weight of the total batch produced. Further encompassed within this invention are dentifrice formulations comprising such materials. The gel/precipitate silica composite for use in a dentifrice composition has a maximum light transmission of at least 25%, preferably at least 40%, within a refractive index range of from about 1.432 to about 1.455; a relative flavor availability as compared to silica sand of at least 50%; a CTAB of less than about 40; and, when incorporated into a dentifrice composition in an amount of 20% by weight, the dentifrice has a RDA value of at most 130, preferably at most 120; a PCR:RDA ratio of from 0.7 to 1.3; and a haze value after 24 hours of less than about 50%.

The essential as well as optional components of the compositions and related methods of making same of the present invention will now be described in more detail.

The gel/precipitate silica composites of the present invention are prepared according to the following two-stage process with a silica gel being formed in the first stage and precipitated silica formed in the second stage. In this process, an aqueous solution of an alkali silicate, such as sodium silicate, is charged into a reactor equipped with mixing means adequate to ensure a homogeneous mixture, and the aqueous solution of an alkali silicate in the reactor is preheated to a temperature of between about 40° C. and about 90° C. and maintained. Preferably, the aqueous alkali silicate solution has an alkali silicate concentration of approximately 3.0 to 35 wt %, preferably from about 3.0 to about 25 wt %, and more preferably from about 3.0 to about 15 wt %. Preferably, the alkali silicate is a sodium silicate with a SiO2:Na2O ratio of from about 1 to about 4.5, more preferably from about 1.5 to about 3.4. The quantity of alkali silicate charged into the reactor is about 10% to 60% by volume of the total silicate used in the batch. An electrolyte, such as sodium sulfate solution, is added to the reaction medium (silicate solution or water) at this point.

Next, an aqueous acidulating agent or acid, such as sulfuric acid, hydrochloric acid, nitric acid, phosphoric acid, and so forth (preferably sulfuric acid), added as a dilute solution thereof (e.g., at a concentration of between about 4 to 35 wt %, more typically about 9.0 to 15.0 wt %) is added to the silicate to form a gel. Once the silica gel is produced and the pH adjusted to the desired level, such as between about 3 and 10, the acid addition is stopped and the gel is adjusted to the reaction temperature, preferably between about 65° C. to about 100° C.

It is important to note that after this first stage is completed, the produced silica gel may be subjected to high shear conditions to modify the gel from its initially produced form. Such high shear conditioning may be performed in any known manner, such as by increased flow rate of liquids, physical mixing in a blending setting, and the like. High shear conditioning is met simply by the modification of the gel component after initial production. Such modification could be measured by a reduction in the average particle size of the gel material after such high shear treatment is undertaken. The resultant gel is otherwise not washed, purified, or cleaned, in any other manner prior to commencement of the second stage.

Next, the second stage begins after the gel reaction temperature is increased, and optionally, additional electrolyte is added to the reactor at this point. Then there is a simultaneous addition to the reactor of (all while the shear rate remains at substantially the same level throughout): (1) an aqueous solution of an acidulating agent previously used and (2) additional amounts of an aqueous solution containing an alkali silicate as is in the reactor, the aqueous solution being preheated to a temperature of about 65° C. to about 100° C. The rate of acidulating agent and silicate additions can be adjusted to control the simultaneous addition pH during the second stage reaction. In addition to the high shear conditions present already, high shear recirculation may be utilized, and the acid solution addition continues until the reactor batch pH drops to between about 3 to about 10.

After the inflows of the acidulating agent and the alkali silicate are stopped, the reactor batch is allowed to age or “digest” for 5 minutes or more, typically 10 to 45 minutes, with the reactor contents being maintained at a constant pH. After the completion of digestion, the high shear mixing, etc., is curtailed, and the resultant reaction batch is filtered and washed with water to remove excess by-product inorganic salts until the wash water from the silica filter cake results in at most 5% salt byproduct content as measured by conductivity.

The silica filter cake is slurried in water, and then dried by any conventional drying techniques, such as spray drying, to produce amorphous silica containing from about 3 wt % to about 50 wt % of moisture. The silica may then be milled to obtain the desired median particle size of between about 3 μm to 25 μm, preferably between about 3 μm to about 20 μm. Classification of even narrower median particle size ranges may aid in providing increased cleaning benefits as well.

As mentioned above, an electrolyte is used during the gel formation, or at both gel formation and precipitate formation as mentioned above. Any suitable electrolyte may be used, with sodium sulfate particularly preferred. When the electrolyte is added during the gel formation step it is introduced at a concentration of about 0.5% to about 2.5% (based on the total batch aqueous solution). The electrolyte may also be directly premixed with one of the process ingredients preliminary to being added to the reaction, for example the electrolyte may be premixed with the sodium silicate. In another alternative embodiment, the electrolyte may be continuously metered into the reaction.

In addition to the above-described production process methodologies of precipitating the synthetic amorphous silicas, the preparation of the silica products is not necessarily limited thereto and it also can be generally accomplished in accordance with the methodologies described, for example, in prior U.S. Pat. Nos. 3,893,840, 3,988,162, 4,067,746, 4,340,583, and 5,891,421, all of which are incorporated herein by reference, as long as such methods are appropriately modified to incorporate the electrolyte addition. As will be appreciated by one skilled in the art, reaction parameters which affect the characteristics of the resultant gel/precipitate silica composite include: the rate and timing at which the various reactants are added; the levels of concentration of the various reactants; the reaction pH; the reaction temperature; the agitation of the reactants during production; and/or the rate at which any electrolytes are added.

Alternative methods of production for this inventive material include in slurry form such as, without limitation, procedures taught within U.S. Pat. No. 6,419,174, to McGill et al., as well as filter press slurry processes as described within and throughout U.S. Pat. No. 6,860,913 to Huang.

The inventive in situ generated composites (also referred to as “combinations”) of silica gel and precipitate are useful as high-cleaning, dental abrasives with correlative lower abrasiveness (with low RDA measurements of at most about 130, for instance, and as low as about 70). The in situ process of this invention has thus surprisingly yielded, with degrees of selectivity followed in terms of reaction pH, reactant concentrations, amount of gel component, high shear production conditions, and, as a result, overall structure of the resultant gel/precipitate silica composite materials made there from, a method for producing a mid-range product (relatively high, cleaning levels with lower abrasion levels) composites. Thus, selection of differing concentrations, pH levels, ultimate gel proportions, among other things, can produce gel/precipitate silica composite materials of mid-range cleaning abrasives in order to accord relatively high pellicle film cleaning results, with lower abrasive properties as compared with the high cleaning materials described above.

For this cleaning material, the gel component is present in an amount between 5% and 60% by weight of the ultimately formed gel/precipitate silica composite material (and thus the precipitated silica component is present in an amount of from 40% to 95% by weight as a result). It is important to note, however, due to the nature of the gel/precipitate composite and its making process, that the percentages noted above are merely best estimates, rather than concrete determination of final amounts of components.

Generally, it has been determined that such specific mid-range cleaning abrasives may be produced through a method of admixing a suitable acid and a suitable silicate material (wherein the acid concentration, in aqueous solution, is from 5 to 25%, preferably from 10 to 20%, and more preferably from 10 to 12%, and the concentration of the silicate starting material is from 4 to 35%, also within an aqueous solution), to initially form a silica gel.

Subsequent to gel formation, sufficient silicate and acid are added to the formed gel for further production of appropriately structured precipitated silica component desired for a mid-range cleaning composite material to be formed. The pH of the overall reaction may be controlled anywhere within the range of 3 to 10. Depending on the amount of gel initially formed, the amount and structure of precipitated silica component may be targeted. It has been realized that in order to provide a mid-range cleaning, low abrasive material through this process, the amount of the gel present during the production is from 10% to 60% by volume of the batch (preferably from 20% to 33%) and the amount of precipitated silica is from 40% to 90% by volume of the batch (preferably from 67% to 80%).

Broadly, the inventive mid-range cleaning gel/precipitated silica combination generally have the following properties within a test dentifrice formulation (as presented below within the examples): RDA (Relative Dentin Abrasion) values of at most 130, preferably between about 80 to about 120, with a ratio of PCR to RDA within the range of 0.7 to 1.3.

The gel/precipitated silica composites of the present invention exhibit oil absorption values in the range of about 30 to about 120, preferably about 40 to about 110, more preferably about 50 to about 90, still more preferably about 60 to about 80.

The gel/precipitated silica composites of the present invention have CTAB values less than about 40, preferably within the range of about 9 to about 35, preferably about 12 to about 25. Similarly, the gel/precipitated silica combination also have improved optical and clarity properties, such as maximum light transmission of at least 25%, preferably at least 40% within a refractive index of from about 1.432 to about 1.455. Additionally, with respect to optical performance, the gel/precipitated silica combination has an index of refraction that is sufficiently low, such that the silica can be included in a transparent toothpaste composition having a relatively high concentration of water. Such index is in the range of about 1.432 to about 1.455, preferably about 1.435 to about 1.445.

Further, the gel/precipitate silica composite materials have relative flavor availability as compared to silica sand of at least 50%, preferably at least 75% and more preferably at least 85%.

The inventive in situ generated gel/precipitate silica composite materials described herein may be utilized alone as the cleaning agent component provided in the dentifrice compositions of this invention, or as an additive with other abrasive materials therein. A mixture of the inventive composite materials with other abrasives physically blended therewith within a suitable dentifrice formulation is potentially preferred in this regard in order to accord targeted dental cleaning and abrasion results at a desired protective level. Thus, any number of other conventional types of abrasive additives may be present in combination with the inventive silica within dentifrices in accordance with this invention.

Other such abrasive particles include, for example, and without limitation, precipitated calcium carbonate (PCC), ground calcium carbonate (GCC), dicalcium phosphate or its dihydrate forms, silica gel (by itself, and of any structure), amorphous precipitated silica (by itself, and of any structure as well), perlite, titanium dioxide, calcium pyrophosphate, hydrated alumina, calcined alumina, insoluble sodium metaphosphate, insoluble potassium metaphosphate, insoluble magnesium carbonate, zirconium silicate, aluminum silicate, chalk, bentonite, particulate thermosetting resins and other suitable abrasive materials known to a person of ordinary skill in the art.

The gel/precipitate silica combination described above, when incorporated into dentifrice compositions as an abrasive, is present at a level of from about 5% to about 50% by weight, more preferably from about 10% to about 35% by weight, particularly when the dentifrice is a toothpaste. Overall dentifrice or oral cleaning formulations incorporating the abrasive compositions of this invention conveniently can comprise the following possible ingredients and relative amounts thereof (all amounts in wt %):

| Ingredient | Amount |
|---|---|
| Liquid Vehicle: | |
| humectant(s) (total) | 5-70 |
| deionized water | 5-70 |
| binder(s) | 0.5-2.0 |
| anticaries agent | 0.1-20 |
| chelating agent(s) | 0.4-10 |
| silica thickener | 3-15 |
| surfactant(s) | 0.5-2.5 |
| all abrasives | 10-50 |
| sweetening agent | <1.0 |
| coloring agents | <1.0 |
| flavoring agent | <5.0 |
| preservative | <0.5 |

In addition, as noted above, the inventive abrasive could be used in conjunction with other abrasive materials, such as precipitated silica, silica gel, dicalcium phosphate, dicalcium phosphate dihydrate, calcium metasilcate, calcium pyrophosphate, alumina, calcined alumina, aluminum silicate, precipitated and ground calcium carbonate, chalk, bentonite, particulate thermosetting resins and other suitable abrasive materials known to a person of ordinary skill in the art.

In addition to the abrasive component, the dentifrice may also contain one or more organoleptic enhancing agents. Organoleptic enhancing agents include humectants, sweeteners, surfactants, flavorants, colorants and thickening agents, (also sometimes known as binders, gums, or stabilizing agents). Humectants serve to add body or “mouth texture” to a dentifrice as well as preventing the dentifrice from drying out. Suitable humectants include polyethylene glycol (at a variety of different molecular weights), propylene glycol, glycerin (glycerol), erythritol, xylitol, sorbitol, mannitol, lactitol, and hydrogenated starch hydrolyzates, as well as mixtures of these compounds. Typical levels of humectants are from about 20 wt % to about 30 wt % of a toothpaste composition.

Sweeteners may be added to the toothpaste composition to impart a pleasing taste to the product. Suitable sweeteners include saccharin (as sodium, potassium or calcium saccharin), cyclamate (as a sodium, potassium or calcium salt), acesulfame-K, thaumatin, neohisperidin dihydrochalcone, ammoniated glycyrrhizin, dextrose, levulose, sucrose, mannose, and glucose.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the product. Suitable colorants are selected from colorants approved by appropriate regulatory bodies such as the FDA and those listed in the European Food and Pharmaceutical Directives and include pigments, such as TiO2, and colors such as FD&C and D&C dyes.

Thickening agents are useful in the dentifrice compositions of the present invention to provide a gelatinous structure that stabilizes the toothpaste against phase separation. Suitable thickening agents include silica thickener; starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; as well as mixtures of these compounds. Typical levels of thickening agents or binders are from about 0 wt % to about 15 wt % of a toothpaste composition.

Therapeutic agents are optionally used in the compositions of the present invention to provide for the prevention and treatment of dental cares, periodontal disease and temperature sensitivity. Examples of therapeutic agents, without intending to be limiting, are fluoride sources, such as sodium fluoride, sodium monofluorophosphate, potassium monofluorophosphate, stannous fluoride, potassium fluoride, sodium fluorosilicate, ammonium fluorosilicate and the like; condensed phosphates such as tetrasodium pyrophosphate, tetrapotassium pyrophosphate, disodium dihydrogen pyrophosphate, trisodium monohydrogen pyrophosphate, tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tanase, and proteases; quarternary ammonium compounds, such as benzalkonium chloride (BAC), benzethonium chloride (BTC), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may also be optionally added to the compositions of the present invention to prevent bacterial growth. Suitable preservatives approved for use in oral compositions such as methylparaben, propylparaben and sodium benzoate may be added in safe and effective amounts.

The dentifrices disclosed herein may also a variety of additional ingredients such as desensitizing agents, healing agents, other caries preventative agents, chelating/sequestering agents, vitamins, amino acids, proteins, other anti-plaque/anticalculus agents, opacifiers, antibiotics, anti-enzymes, enzymes, pH control agents, oxidizing agents, antioxidants, and the like.

Water provides the balance of the composition in addition to the additives mentioned. The water is preferably deionized and free of impurities. The total amount of water in a dentifrice is usually from about 5 wt % to about 35 wt % of water. Useful silica thickeners for utilization within such a toothpaste formulation include, as a non-limiting example, amorphous precipitated silica such as ZEODENT® 165 silica. Other preferred (though non-limiting) silica thickeners are ZEODENT 163 and/or 167 and ZEOFREE® 153, 177, and/or 265 silicas, all available from J. M. Huber Corporation, Havre de Grace, Md., U.S.A.

For purposes of this invention, a “dentifrice” has the meaning defined in Oral Hygiene Products and Practice, Morton Pader, Consumer Science and Technology Series, Vol. 6, Marcel Dekker, NY 1988, p. 200, which is incorporated herein by reference. Namely, a “dentifrice” is “ . . . a substance used with a toothbrush to clean the accessible surfaces of the teeth. Dentifrices are primarily composed of water, detergent, humectant, binder, flavoring agents, and a finely powdered abrasive as the principal ingredient . . . a dentifrice is considered to be an abrasive-containing dosage form for delivering anti-caries agents to the teeth.” Dentifrice formulations contain ingredients which must be dissolved prior to incorporation into the dentifrice formulation (e.g. anticaries agents such as sodium fluoride, sodium phosphates, flavoring agents such as saccharin).

The various silica and toothpaste (dentifrice) properties described herein were measured as follows, unless indicated otherwise. The external surface area of silica is determined by adsorption of CTAB (cetyltrimethylammonium bromide) on the silica surface, the excess separated by centrifugation and determined by titration with sodium lauryl sulfate using a surfactant electrode. Specifically, about 0.5 g of silica is accurately weighed and placed in a 250-ml beaker with 100.00 ml CTAB solution (5.5 g/L, adjusted to pH 9.0±0.2), mixed on an electric stir plate for 30 minutes, then centrifuged for 15 minutes at 10,000 rpm. One ml of 10% TRITON X-100® is added to 5 ml of the clear supernatant in a 100-ml beaker. The pH is adjusted to 3.0-3.5 with 0.1 N HCl and the specimen is titrated with 0.0100 M sodium lauryl sulfate using a surfactant electrode (Brinkan SURI501-DL) to determine the endpoint. The CTAB value is then calculated from the difference between CTAB stock solution and the sample solution after absorption.

The oil absorption values are measured using the rub out method as described in ASTM D281. This method is based on a principle of mixing linseed oil with silica by rubbing with a spatula on a smooth surface until a stiff putty-like paste is formed. By measuring the quantity of oil required to have a paste mixture which will curl when spread out, one can calculate the oil absorption value of the silica--the value which represents the volume of oil required per unit weight of silica to saturate the silica sorptive capacity. A higher oil absorption level indicates a higher structure of precipitated silica; similarly, a low value is indicative of what is considered a lower structure precipitated silica. Calculation of the oil absorption value was done as follows:

$$\begin{matrix}Oil absorption = \frac{ml oil absorbed}{weight of silica , grams} \times 100 \\ = \frac{ml oil}{100 gram silica}\end{matrix}$$

Median particle size is determined using a Model LA-300 or an equivalent laser light scattering instrument available from Horiba Instruments, Boothwyn, Pa.

The % 325 mesh residue of the inventive silica is measured utilizing a U.S. Standard Sieve No. 325, with 44 micron or 0.0017 inch openings (stainless steel wire cloth) by weighing a 10.0 gram sample to the nearest 0.1 gram into the cup of the 1 quart Hamilton mixer Model No. 30, adding approximately 170 ml of distilled or deionized water and stirring the slurry for at least 7 min. Transfer the mixture onto the 325 mesh screen; wash out the cup and add washings onto the screen. Adjust water spray to 20 psi and spray directly on screen for two minutes (the spray head should be held about four to six inches above the screen cloth). Wash the residue to one side of the screen and transfer by washing into an evaporating dish using distilled or deionized water from a washing bottle. Let stand for two to three minutes and decant the clear water. Dry (convection oven @ 150° C. or under infrared oven for approx. 15 min.) cool and weigh residue on analytical balance.

Moisture or Loss on Drying (LOD) is the measured silica sample weight loss at 105° C. for 2 hours. The pH values of the reaction mixtures (5 weight % slurry) encountered in the present invention can be monitored by any conventional pH sensitive electrode.

Sodium sulfate content was measured by conductivity of a known concentration of silica slurry. Specifically, 38 g silica wetcake (or 13.3 g dry) sample was weighed into a one-quart mixer cup of a Hamilton Beach Mixer, model Number 30, and 140 ml (170 ml for diy sample) of deionized water was added. The slurry was mixed for 5 to 7 minutes, then the slurry was transferred to a 250-ml graduated cylinder and the cylinder filled to the 250-ml mark with deionized water, using the water to rinse out the mixer cup. The sample was mixed by inverting the graduated cylinder (covered) several times. A conductivity meter, such as a Cole Palmer CON 500 Model #19950-00, was used to determine the conductivity of the slurry. Sodium sulfate content was determined by comparison of the sample conductivity with a standard curve generated from known method-of-addition sodium sulfate/silica composition slurries.

The Relative Dentin Abrasion (RDA) values of dentifrices containing the silica compositions used in this invention are determined according to the method set forth by Hefferen, Journal of Dental Res., July-August 1976, 55 (4), pp. 563-573, and described in Wason U.S. Pat. Nos. 4,340,583, 4,420,312 and 4,421,527, which publications and patents are incorporated herein by reference.

The cleaning property of dentifrice compositions is typically expressed in terms of Pellicle Cleaning Ratio (“PCR”) value. The PCR test measures the ability of a dentifrice composition to remove pellicle film from a tooth under fixed brushing conditions. The PCR test is described in “In Vitro Removal of Stain With Dentifrice” G. K. Stookey, et aI., J. Dental Res., 61, 1236-9, 1982. Both PCR and RDA results vary depending upon the nature and concentration of the components of the dentifrice composition. PCR and RDA values are unitless.

Properties relating to the gel toothpaste clarity, such as refractive index and haze were determined as follows:

As a first step in measuring refractive index (“RI”) and degree of light transmission, a range of glycerin/water stock solutions (about 10) was prepared so that the refractive index of these solutions lies between 1.428 and 1.46. The exact glycerin/water ratios needed depend on the exact glycerin used and is determined by the technician making the measurement. Typically, these stock solutions will cover the range of 70 wt % to 90 wt % glycerin in water. To determine refractive index, one or two drops of each standard solution are separately placed on the fixed plate of a refractometer (Abbe 60 Refractometer Model 10450). The covering plate is fixed and locked into place. The light source and refractometer are switched on and the refractive index of each standard solution is read.

Into separate 20-ml bottles, accurately weighed was 2.0±0.01 ml of the inventive gel/precipitate silica product and added was 18.0±0.01 ml of each respective stock glycerin/water solution (for products with measured oil absorption above 150, the test used 1 g of inventive gel/precipitate silica product and 19 g of the stock glycerin/water solution). The bottles were then shaken vigorously to form silica dispersions, the stoppers were removed from the bottles, and the bottles were placed in a desiccator. The desiccator was then evacuated with a vacuum pump (about 24 inches Hg) for 120 minutes and visually inspected for complete de-aeration. The % Transmittance (“% T”) at 590 nm (Spectronic 20 D+) was measured after the samples returned to room temperature (about 10 minutes), according to the instrument manufacturer's operating instructions.

The % Transmittance was measured on the inventive product/glycerin/water dispersions by placing an aliquot of each dispersion in a quartz cuvette and reading the % T at 590 nm wavelength for each sample on a 0-100 scale. The % Transmittance vs. RI of the stock solutions used was plotted on a curve. The refractive index of the inventive product was defined as the position of the plotted peak maximum (the ordinate or X-value) on the % Transmittance vs. the RI curve. The Y value (or abscissa) of the peak maximum was the % Transmittance.

The “% Haze of the clear gel toothpaste is measured by a BYK-Gardner Haze-Gard plus instrument. The Haze-Gard plus is a stationary instrument designed to measure the appearance of glass and of films, packaging and pars made of plastic and other transparent materials. The specimen surface is illuminated perpendicularly, and the transmitted light is measured photoelectrically, using an integrating sphere (0 degree/diffuse geometry). The instrument is first calibrated according to the manufacturer's directions. Next, two microscope slides, having dimensions of 38×75 mm, and a thickness 0.96 to 1.06 mm, are placed on a flat surface. One slide is covered with a Plexiglas spacer, (38×75 mm, 3 mm thickness, with 24×47 mm open area). The gel toothpaste in squeezed into the open area of the Plexiglas spacer. The second slide is placed over the toothpaste and pressure applied, by hand, to eliminate excess toothpaste and air. The sample is placed on the optical port of the precalibrated meter and the haze values are obtained. Lower haze values described toothpastes having greater transparency.

The flavor performance analysis was conducted by gas chromatography/Mass Spectrometry using an Hewlett Packard GC/MS 5890/5972 device. A Gerstel MPS2 with 2.5 ml static headspace syringe was used in the GC/MS. A Stabilwax 60 m chromatography column was used having a 0.25 mm inner diameter and a 0.25 μm film thickness. The flavor tested was spearmint oil, specifically Aldrich no. W30322-4.

The chromatography process parameters were as follows: the syringe temperature was 65° C.; the Agitator temperature was 60° C.; the head pressure was 27 psi.; the split flow was 30 ml/min with a 1 min splitless injection; the injector temperature was 250° C.; the detector temperature was 280° C.; the temperature of the oven was raised from 40° C. to 230° C. at 6° C./min.

The silica samples were dried at 105° C. for 4 hours then equilibrated in a desiccator for 4 hours. 0.5000 g of silica material was metered into a 20 ml vial, and 10 μL of flavor was added to the vial and then the vial was immediately capped. Each sample was vortexed for 10 seconds and allowed to equilibrate overnight. The instrument run was then setup so that each sample was incubated at 60° C. for 60 min with shaking, immediately after which 1 ml of headspace was then injected into the GC/MS.

### EXAMPLES

The invention will now be described in more detail with respect to the following non-limiting examples which were performed with the above described equipment, materials and methods.

### Gel/Precipitated Silica Composite Production

Several examples 1-5 were prepared both according to the present invention (i.e., with sulfate addition) and according to the prior art (without sulfate). In this process, these examples contained 29% by volume gel and thus about 71% by volume precipitated silica.

In a first phase, a silica gel was formed when 174 L of aqueous solution of 6% sodium silicate with a SiO₂:Na₂O ratio of 3.3 was charged into a reactor and agitated therein at a speed of 50 rpm and heated to a temperature of 85° C. For Examples 1 and 2, 10 Kg of anydrous sodium sulfate were added during gel formation. For Example 3, 5 Kg of anhydrous sodium sulfate were added during gel formation. For Example 4 and 5, no electrolyte was added during gel formation. Then 11.4% sulfuric acid was added at a rate 4.09 L/minute for 7 minutes. After 7 minutes, the acid addition was stopped concluding the gel formation stage.

In the second stage, the slurry from the first phase was then heated to a temperature of 93° C., this temperature being maintained throughout the batch. The agitator speed was then increased to 80 rpm. Also, recirculation line flow and a rotor-stator mixer (providing high shear) were started, both at 60 Hz. Precipitate formation followed wherein, for Examples 2 and 5, 10 Kg of anhydrous sodium sulfate were added; for Example 3, 5 Kg of anhydrous sodium sulfate was added; and for Examples 1 and 4, no additional sulfate was added. Precipitated silica was formed by simultaneous addition of acid (at a rate of 3.2 L/minute) and silicate solution (pre-heated to a temperature of 85° C., having a concentration of 16.21% and added at a rate of 8.88 L/min) to the slurry in the reactor. The simultaneous addition continues for a period of 48 minutes. After 48 minutes, silicate flow was stopped. The acid flow continued at a rate of 3.2 L/minute until the pH dropped to 7.0 at which point the acid flow was reduced to 1 L/minute. Acid flow was continued at 1 L/minute until the pH approached 5.3-5.5. Then the acid flow was stopped and the batch digested for 10 minutes while being maintained at a temperature of 93° C., during which the pH was maintained between 5.3 and 5.5.

The resultant slurry was then recovered by filtration, washed to a sodium sulfate concentration of less than about 5% (preferably less than 4%, and most preferably below 2%) and then spray dried to a level of about 5% moisture. The dried product was then milled to uniform size. As mentioned above, five different samples were prepared according to the above procedure, with three prepared according to the present invention (Examples 1-3, making use of sulfate salt) and two comparative examples, one that contained no salt (Example 4) and on that contained no salt in the gel formation phase (Example 5). Several properties of these materials were then measured and the results are set forth in Table 1, below.

TABLE 1
Inventive and Comparative Silica Physical and Chemical Characteristics

| | | | | Ex. 4 | Ex. 5 |
|---|---|---|---|---|---|
| Physical Test | Ex. 1 | Ex. 2 | Ex. 3 | Comparative | Comparative |
| Electrolyte | Gel | Gel & Precipitate | Gel & | No | Precipitate |
| Addition | Formation | Formation | Precipitate | Electrolyte | Formation |
| | | | Formation | | |
| % Moisture | 4.4 | 4.7 | 4.3 | 4.2 | 5.1 |
| 325 Mesh | 1.46 | 0.73 | 0.61 | 0.52 | 2.54 |
| Residue % | | | | | |
| CTAB Surface | 17 | 15 | 21 | 65 | 46 |
| Area m²/g | | | | | |
| Median Particle | 12.55 | 11.40 | 12.64 | 14.00 | 15.29 |
| Size (μm) | | | | | |
| Na₂SO₄ % | 1.22 | 0.51 | 0.90 | 0.35 | 2.24 |
| Oil Absorption | 70 | 63 | 81 | 95 | 93 |
| mL/100 g | | | | | |
| pH 5% | 7.39 | 7.67 | 7.58 | 7.40 | 7.18 |
| % T (10% | 51.6 | 47.2 | 77.0 | 77.0 | 75.4 |
| Glycerin Test) | | | | | |
| % T max at R.I. | 1.435 | 1.438 | 1.438 | 1.445 | 1.445 |

Flavor retention tests were performed according to the procedure described previously. Silica sand (SIL-CO-SIL® 63, US Silica Company) was tested as a reference material.

TABLE 2
Flavor Retention Comparisons

| Example | % Available Flavor |
|---|---|
| Silica sand | 100 |
| Example 1 Silica | 93 |
| Example 2 Silica | 92 |
| Example 3 Silica | 92 |
| Example 4 Silica | 37 |
| Example 5 Silica | 34 |

As can be seen in Table 2, the silicas prepared according to the present invention offer excellent flavor retention performance, comparable to silica sand.

### Dentifrice Formulation Examples

Toothpaste-dentifrice formulations were then prepared incorporating the silica materials set forth in Table 1. To prepare the dentifrices, the glycerin, sodium carboxymethyl cellulose, polyethylene glycol and sorbitol were mixed together and stirred until the ingredients were dissolved to form a first admixture. The deionized water, sodium fluoride, and sodium saccharin were also mixed together and stirred until these ingredients are dissolved to form a second admixture. These two admixtures were then combined with stirring. Thereafter, the optional color was added with stirring to obtain a “pre-mix”. The pre-mix was placed in a Ross mixer (Model DPM-1) and silica thickener, abrasive silica and titanium dioxide were mixed in without vacuum. A 30-inch vacuum was drawn and the resultant admixture was stirred for approximately 15 minutes. Lastly, sodium lauryl sulfate, color, and flavor were added and the admixture was stirred for approximately 5 minutes at a reduced mixing speed. The resultant dentifrice was transferred to plastic laminate toothpaste tubes and stored for future testing. Four different dentifrice formulations, each using one of the abrasive Examples 1-4 set forth above were prepared according to the formula shown in Table 3 below. The dentifrice formulation utilized was considered a suitable test dentifrice formulation for the purposes of determining PCR and RDA measurements for the inventive and comparative cleaning abrasives.

TABLE 3
Dentifrice Components

| Component | Proportion |
|---|---|
| Glycerin (99.7%), % | 10 |
| Sorbitol (70%), % | 48.26 |
| Deionized water, % | 13.0 |
| CARBOW AX ® 600¹ (PEG-12), % | 3.0 |
| CEKOL ® 2000 CMC², % | 1.0 |
| Sodium Saccharin, % | 0.2 |
| Sodium Fluoride, % | 0.240 |
| Silica thickener ZEODENT ® 165³, % | 2.0 |
| Abrasive (selected from Table 1 materials), % | 20.0 |
| Color, Blue 1.0% solution, % | 0.1 |
| Sodium lauryl sulfate, % | 1.20 |
| Flavor, % | 1.0 |
| Total | 100.000 |

¹A polyethylene glycol available from Dow Chemical Company, Midland, MI
²A carboxymethylcellulose available from CP Kelco Oy, Aanekoski, Finland
³An amorphous, precipitated high structure silica thickening available from J. M. Huber Corporation, Havre de Grace, MD

Several dentifrice formulations were prepared using the dentifrice formulation of Table 3 including the different silica abrasives as indicated in Table 4.

TABLE 4
Different Inventive and Comparative Dentifrice Formulations

Dentifrice
Formulation No.

| Silica Abrasive from Table 1, % | 1 | 2 | 3 | 4 |
|---|---|---|---|---|
| Example 1 | 20 | 0 | 0 | 0 |
| Example 2 | 0 | 20 | 0 | 0 |
| Example 3 | 0 | 0 | 20 | 0 |
| Example 4 (Comparative) | 0 | 0 | 0 | 20 |

These dentifrices were then evaluated for PCR and RDA properties and haze value, according to the methods described above. The results for each dentifrice formulation are provided in Table 5 below. Formulations 1-3, below are directed to the present invention and Formulation 4 is comparative.

TABLE 5
Dentifrice Formulation Physical Testing Results

| Dentifrice | | | | % Haze Value |
|---|---|---|---|---|
| Formulation | PCR | RDA | PCR:RDA | (24 Hours) |
| 1 | 96 | 108 | 0.88 | 47 |
| 2 | 90 | 105 | 0.85 | 48 |
| 3 | 80 | 87 | 0.92 | 42 |
| 4 (Comparative) | 92 | 89 | 1.03 | 67 |

The data in the above tables demonstrate that while the silica of the present invention are not superior in every performance category, they offer a very desirable functional performance profile including good cleaning, low abrasivity, improved flavor compatibility, and a relatively high degree of transmittance, even at an index of refraction that is sufficiently low so that the silica can be included in a transparent toothpaste composition having a relatively high concentration of water. It must be particularly emphasized that the silica of the present invention exhibits outstanding flavor compatibility performance.

It will be appreciated by those skilled in the art that changes could be made to the embodiments described above without departing from the broad inventive concept thereof. It is understood therefore that this invention is not limited to the particular embodiments disclosed, but it is intended to cover modifications within the spirit and scope of the present invention as defined by the appended claims.

We claim:

1. A gel/precipitate silica composite for use in a dentifrice composition, wherein said composite has a maximum light transmission of at least 25% within a refractive index range of from about 1.432 to about 1.455; a relative flavor availability as compared to silica sand of at least 50%; a CTAB of less than about 40; and, when incorporated into a dentifrice composition in an amount of 20% by weight, said dentifrice has a RDA (Relative Dentin Abrasion) value of at most 130; a PCR (Pellicle Cleaning Ratio):RDA ratio of from 0.7 to 1.3; and a haze value after 24 hours of less than about 50%.

2. The gel/precipitate silica composite of claim 1 wherein the maximum light transmission of the composite is at least 40%.

3. The gel/precipitate silica composite of claim 1 wherein the refractive index range is from about 1.435 to about 1.445.

4. The gel/precipitate silica composite of claim 1 wherein the maximum light transmission of the composite is at least 40% and the refractive index range is from about 1.435 to about 1.445.

5. The gel/precipitate silica composite of claim 1 wherein the relative flavor availability as compared to silica sand is at least 75%.

6. The gel/precipitate silica composite of claim 1 wherein the relative flavor availability as compared to silica sand is at least 85%.

7. The gel/precipitate silica composite of claim 1 wherein the CTAB of the composite is from about 9 to about 35.

8. The gel/precipitate silica composite of claim 1 wherein the CTAB of the composite is from about 12 to about 25.

9. The gel/precipitate silica composite of claim 1 wherein said dentifrice has a RDA of at most 120.

10. The gel/precipitate silica composite of claim 1 wherein said dentifrice exhibits a PCR:RDA ratio of from 0.8 to 1.0.

11. A dentifrice comprising the gel/precipitate composite of any one of claims 1 to 10.

12. A method of producing a gel/precipitate silica composite for use in a dentifrice composition, wherein said composite has a maximum light transmission of at least 25% within a refractive index range of from about 1.432 to about 1.455; a relative flavor availability as compared to silica sand of at least 50%; a CTAB of less than about 40; and, when incorporated into a dentifrice composition in an amount of 20% by weight, said dentifrice has a RDA value of at most 130; a PCR:RDA ratio of from 0.7 to 1.3; and a haze value after 24 hours of less than about 50%, said method comprising the sequential steps of
a. admixing an electrolyte, an alkali silicate. and an acidulating agent to form a silica gel in a reaction medium; and, without first washing, modifying, or purifying said silica gel,
b. subsequently introducing to said reaction medium comprising said silica gel of step (a) a sufficient amount of an alkali silicate and an acidulating agent to form a precipitated silica, thereby producing a gel/precipitate silica composite.

13. The method of claim 12, wherein subsequent to step (a), the reaction medium is subjected to high shear conditions.

14. The method of claim 12, wherein the electrolyte is an alkali metal salt or an alkaline earth metal salt.

15. The method of claim 12, wherein the electrolyte is sodium sulfate.

16. The method of claim 12, wherein step (a), the electrolyte is introduced at a weight ratio of concentration of about 0.5% to about 2.5% based on the total batch aqueous solution.

17. The method of claim 12, wherein an electrolyte is introduced in step (b).

18. The method of claim 17, wherein the electrolyte of step (b) is sodium sulfate.

19. A method of producing a gel/precipitate silica composite for use in a dentifrice composition, wherein said composite has a maximum light transmission of at least 25% within a refractive index range of from about 1.432 to about 1.455; a relative flavor availability as compared to silica sand of at least 50%; a CTAB of less than about 40; and, when incorporated into a dentifrice composition in an amount of 20% by weight, said dentifrice has a RDA value of at most 130; a PCR:RDA ratio of from 0.7 to 1.3; and a haze value after 24 hours of less than about 50%, said method comprising the sequential steps of
a. admixing an electrolyte, an aqueous solution of an alkali silicate having a concentration of from about 3% to about 35%, and an aqueous solution of an acidulating agent having an acid concentration of from about 4% to about 35% together at a temperature from about 40 to about 90° C. and under agitation to form a silica gel in a reaction medium; and, without first washing, modifying, or purifying said silica gel,
b. subsequently introducing to said reaction medium comprising said silica gel of step (a) a sufficient amount of an alkali silicate and an acidulating agent to form a precipitated silica, thereby producing a gel/precipitate silica composite, wherein the pH of the overall reaction is within the range of from 3 to 10.

20. The method of claim 19, wherein subsequent to step (a), said reaction medium is subjected to high shear conditions.
